(19) United States
(12) Patent Application Publication
(10) Pub. No.: US 2007/0105100 A1
(43) Pub. Date: May 10, 2007

(54) Process for assay of nucleic acids by competitive hybridization using a dna microarray

(75) Inventors: Hiroto Yoshii, Tokyo (JP); Nobuko Yamamoto, Kanagawa-ken (JP); Keiko Yonezawa, Kanagawa-ken (JP)

Correspondence Address: FITZPATRICK CELLA HARPER & SCINTO, 30 ROCKEFELLER PLAZA, NEW YORK, NY, 10112, US

(73) Assignee: CANON KABUSHIKI KAISHA, Tokyo (JP)

(21) Appl. No.: 10/546,429

(22) PCT Filed: Apr. 28, 2004
(86) PCT No.: PCT/JP04/06214

§ 371 Date: Jul. 5, 2006

(30) Foreign Application Priority Data
Apr. 28, 2003 (JP) 2003-124024
May 21, 2003 (JP) 2003-143465
Jun. 17, 2003 (JP) 2003-172030

(51) Int. Cl. C12Q 1/68

(52) U.S. Cl. 435/6

(57) ABSTRACT

A process for rapid and simple assay of nucleic acid on the basis of competitive hybridization using a DNA microarray that comprises a step of hybridizing a labeled nucleic acid other than the target nucleic acid with one of the immobilized probes on the substrate, where the labeled nucleic acid has a known sequence complementary to the probe and can bind thereto specifically.

### TECHNICAL FIELD

The present invention relates to a process for treating immobilized nucleic acid probes such as DNA microarray. It also relates to a process for detecting a target nucleic acid using this treating process. Furthermore, it relates to a process for determining nucleic acid concentration, which enables readily estimation of the concentration of a target nucleic acid in a sample. Particularly, the present invention relates to an assay process suitably applicable when using a DNA microarray having immobilized oligonucleotides on a substrate.

### BACKGROUND ART

There are systems to determine expression or sequence of a gene using a DNA microarray (for example, Japanese Patent Application Laid-Open Nos. H10-272000 and H11-187900). In these systems, oligonucleotide probes or cDNA probes arrayed on a substrate are hybridized with a sample DNA, for example, a sample derived from a living organism, in an assay solution on the substrate.

In the above system, the oligonucleotide probe or the cDNA probe is not labeled but the nucleic acid in the assay solution is labeled. Thus analysis of the nucleic acid in the assay solution is carried out on the principle that only the site(s) of the probe where hybridization has occurred is specified with the labeled substance.

The conventional processes except for the electric detection method, however, require introduction of a certain labeling substance to a sample nucleic acid to be studied. Radioisotopes have been used for labeling, but because of hazardous nature thereof, fluorescent substances are now commonly used for labeling. There is also a process in which a labeling substance is not directly introduced into a sample but a reactive group such as amino group is introduced into the sample, through which a labeling substance is covalently bonded to introduce a labeling substance into the sample.

In any of these processes, introducing labeling into the sample is a laborious, time-consuming and cost-incurring step. Furthermore it may result in unstable quantitativeness of the final hybridization detection.

To overcome these problems, there is a method where hybridization is detected by an electric measurement utilizing an intercalator. This method, however, cannot utilize inexpensive DNA microarrays which allow high integration.

### DISCLOSURE OF THE INVENTION

The present invention provides a new process, which saves time and labor in use of a probe array for assay. The process of the present invention for treating probe nucleic acids immobilized at predetermined positions on a substrate for nucleic acid detection is characterized in a step of treating the immobilized nucleic acid on the substrate with a labeled known nucleic acid other than the nucleic acid to be detected (hereinafter referred to a target nucleic acid), where the labeled known nucleic acid contains a complementary sequence and can specifically bind to one of the immobilized probe nucleic acid on the above-described substrate. The known nucleic acid is desirably an artificial nucleic acid.

The present invention also provides a detection process using the above-described treating process. That is, a process for detecting a nucleic acid using a nucleic acid probe being a specified nucleic acid immobilized at a known position on a substrate, the process comprising the steps of:

preparing a labeled nucleic acid of a known sequence which contains a sequence complementary to the probe nucleic acid and can specifically bind to the probe, the labeled nucleic acid being not a target nucleic acid to be detected;

treating the immobilized probe on the substrate with the labeled nucleic acid to make the labeled nucleic acid bind to the probe

reacting a target nucleic acid with the probe on the substrate; and

detecting an amount of the label nucleic acid attached to the immobilized probe.

The present invention also provides a process for detecting a target nucleic acid comprising the steps of:

providing a nucleic acid probe being a predetermined nucleic acid immobilized at a predetermined position on a substrate;

providing a solution containing a sample nucleic acid and a labeled nucleic acid of a known sequence which contains a sequence complementary to the probe nucleic acid and can specifically bind to the probe, the labeled nucleic acid being not a target nucleic acid to be detected;

contacting the solution to the probe to allow binding of the nucleic acids in the solution to the immobilized probe; and

detecting an amount of the labeled nucleic acid attached to the immobilized probe nucleic acid,

wherein the target nucleic acid bound to the probe is detected based on the detected amount of the labeled nucleic acid.

By using such an assay process, nucleic acid can be detected by hybridization reaction using a highly integrated and cheap DNA microarray, which saves time and labor.

The present invention also provides a process for analyzing a nucleic acid concentration using an immobilized probe nucleic acid being a specific nucleic acid immobilized at a predetermined position on a substrate, the process comprising the steps of

(1) preparing a solution containing a labeled nucleic acid of a known sequence which contains a sequence complementary to the probe nucleic acid and can specifically bind to the probe in a predetermined concentration and also a nucleic acid derived from a sample;

(2) contacting the solution with the immobilized probe for hybridization; and

(3) detecting an amount of the labeled nucleic acid attached to the probe,

wherein a concentration of the sample-derived nucleic acid in the solution is estimated based on a decrease in the amount of the labeled nucleic acid attached to the probe obtained in the step (3) compared to an amount of the labeled nucleic acid attached to the probe when hybridization is carried out with a solution containing the labeled nucleic acid at a predetermined concentration but not containing the nucleic acid derived from the sample.

By using such an assay process, quantitative analysis of the target nucleic acid, i.e., target substance, can be performed with hybridization reaction which saves time and labor using a highly integrated and cheap DNA microarray.

The present invention also provides a process for analyzing a nucleic acid concentration using an immobilized probe nucleic acid being a specific nucleic acid immobilized at a predetermined position on a substrate, wherein the process comprises steps of:

introducing a first solution containing a target nucleic acid to be determined derived from a sample into a chamber in which the immobilized probe nucleic acid has been arranged;

introducing a second solution which contains a labeled nucleic acid of a known sequence which contains a sequence complementary to the probe nucleic acid and can specifically bind to the probe so that the both solution may be mixed with each other; and

detecting the amount of the label of the labeled nucleic acid bound to the immobilized probe nucleic acid, wherein

the concentration of the target nucleic acid in the mixed solution is estimated based on the correlation between the amount of the second solution introduced into the chamber and a change in the amount of the label attached to the immobilized probe after the introduction of the second solution.

By using such an assay process, quantitative analysis of the target nucleic acid can be performed more accurately by hybridization reaction which saves time and labor using a highly integrated and cheap DNA microarray.

The present invention also provides a kit for detecting a target nucleic acid in a sample comprising:

an immobilized nucleic acid probe being a specific nucleic acid immobilized at a predetermined position on a substrate; and

a solution containing a labeled nucleic acid of a known sequence which contains a sequence complementary to the probe nucleic acid and can specifically bind to the probe,

wherein the solution is used as a mixture with a solution containing the sample.

Such an invention effectively enables a hybridization experiment to be performed rapidly and easily using a highly integrated and cheap DNA microarray. It also effectively enables a highly quantitative experiment to be performed without incurring cost.

The present invention also provides an immobilized probe nucleic acid for nucleic acid detection wherein a probe nucleic acid is immobilized at a predetermined position on a substrate and a labeled nucleic acid, to which a complementary nucleic acid specifically bindable to the probe nucleic acid has been bound by hybridization.

In this case, preferably 60% or more of the probe nucleic acids immobilized on the substrate are binding the above-described labeled nucleic acids. The content of the labeled nucleic acids is, however, not essential to the principle of this process, and even if the percent of the probes bound to the labeled nucleic acids is less than 60%, the purpose can be attained.

The present invention also provides a process for detecting a nucleic acid comprising the steps of:

providing an immobilized probe nucleic acid being a specific nucleic acid immobilized at a predetermined position on a substrate, to which a labeled nucleic acid of a known sequence which contains a sequence complementary to the probe nucleic acid and can specifically bind to the probe is binding by hybridization at a predetermined amount;

contacting a solution containing a target nucleic acid with the immobilized probe nucleic acid for hybridization; and

determining an amount of the labeled nucleic acid attached to the immobilized probe nucleic acid, wherein

the target nucleic acid bound to the immobilized probe nucleic acid is determined by comparison of the predetermined amount of the labeled nucleic acid attached to the immobilized probe nucleic acid and the amount of the labeled nucleic acid determined in the determination step.

The present invention also provides a process for analyzing a nucleic acid concentration, wherein the process comprises steps of:

(1) providing an immobilized nucleic acid probe by immobilizing a specific nucleic acid at a predetermined position on a substrate;

(2) binding a labeled nucleic acid, which has a complementary nucleic acid specifically bindable to the immobilized probe nucleic acid, to the immobilized probe nucleic acid by hybridization;

(3) contacting a solution containing a target nucleic acid with the probe for hybridization; and

(4) detecting an amount of the labeled nucleic acid bound to the probe, wherein the concentration of the nucleic acid derived from the sample in the solution is estimated by comparison of an amount of the labeled nucleic acid bound to the probe in the preliminary hybridization step (2) and the amount of the labeled nucleic acid detected in the detection step (3).

According to such an invention, presence of the probe nucleic acids immobilized on the substrate can be confirmed by the labeled nucleic acid which has been bound by hybridization reaction before shipping or conducting an experiment.

This enables not only to provide an array of high quality at low cost, but also to provide a remarked effect that the quality thereof before detection operation can be verified. Furthermore, since the quantification of the probe DNA on a DNA microarray can be made prior to the hybridization experiment of unknown sample nucleic acid, experiments such as determination of concentration of unknown nucleic acid can be performed more precisely.

Furthermore, since labeling of unknown samples is not required in this process, treating of the samples is greatly simplified with an advantage of shortening time. There is also an advantage of improved quantitativity of the experiment system using DNA microarray.

The present invention also provides a process for analyzing a nucleic acid to determine gene polymorphism comprising steps of:

(1) providing nucleic acid probes by immobilizing nucleic acids corresponding to all alleles of a target gene polymorphism at predetermined positions on a substrate;

(2) contacting a plurality of labeled nucleic acids having complementary sequence to the probes respectively and able to bind thereto specifically, and an unlabeled unknown nucleic acid derived from a sample to the probes for hybridization; and

(3) detecting an amount of the labeled nucleic acid bound to the probe nucleic acids, wherein gene polymorphism of the nucleic acid derived from the sample is determined based on the balance of the amounts of the labeled nucleic acids bound to the probes.

The whole human genome is considered to contain over one million SNPs. Even if limited to important SNP species, the number of SNPs is still enormous. Thus it is extremely difficult to handle them by the conventional electric measuring process.

According to the present invention, a process for analyzing the nucleic acid is provided for sensitive gene polymorphism determination being time and labor saving with high integration.

Other features and advantages of the present invention will be apparent from the following description taken in conjunction with the accompanying drawings, in which like reference characters designate the same or similar parts throughout the figures thereof.

### BRIEF DESCRIPTION OF THE DRAWINGS

The accompanying drawings, which are incorporated in and constitute a part of the specification, illustrate embodiments of the invention and, together with the description, serve to explain the principles of the invention.

FIG. 1 shows an embodiment of the assay process for invention;

FIG. 2 illustrates the competition between a labeled strand complementary with a probe and an unknown nucleic acid A;

FIG. 3 illustrates the competition between the labeled nucleic acid strand complementary to probe and the unknown nucleic acid B;

FIG. 4 shows the relation between the target nucleic acid A and the nucleic acid B;

FIG. 5 shows a result of an experiment with a series of dilution;

FIG. 6 shows competition relationship when the labeled complementary nucleic acid is shorter than a probe;

FIG. 7 is a drawing showing competition when the labeled complementary nucleic acid is longer than the probe;

FIG. 8 is an overview diagram of an observation system using a confocal microscope;

FIG. 9 is an enlargement schematic view of a portion of the hybridization chamber;

FIG. 10 is a flow chart to illustrate the assay process of the present invention;

FIG. 11 schematically shows a DNA microarray after pre-hybridization;

FIG. 12 schematically illustrates an embodiment of the assay process of the present invention;

FIG. 13 schematically illustrates an embodiment of the assay process of the present invention;

FIG. 14 schematically illustrates competitive reaction between the labeled complementary nucleic acid and unknown nucleic acid A;

FIG. 15 schematically shows the relation between the probe and unknown nucleic acid B;

FIG. 16 schematically illustrates a DNA microarray where competition of FIG. 15 occurred;

FIG. 17 schematically shows the relation between target nucleic acid A and sample nucleic acid B;

FIG. 18 is a flow chart of an example procedure of the assay process of the present invention;

FIG. 19 schematically shows the state of a DNA microarray after the step 704 for predetermination of probe density;

FIG. 20 schematically shows the relation between the concentration of labeled complementary nucleic acid in the hybridization chamber and the fluorescence intensity of a spot;

FIG. 21 schematically illustrates competitive reaction occurring in the hybridization step;

FIG. 22 schematically illustrates competitive reaction occurring in the hybridization step;

FIG. 23 schematically illustrates relation between nucleic acid A and nucleic acid B;

FIG. 24 schematically illustrates a result of an experiment using a serial dilution of an unknown nucleic acid sample;

FIG. 25 schematically illustrates competitive reaction when the labeled complementary nucleic acid is shorter than the probe; and

FIG. 26 schematically illustrates competitive reaction when the labeled complementary nucleic acid is longer than the probe.

### BEST MODE FOR CARRYING OUT THE INVENTION

Preferred embodiments of the present invention will now be described in detail referring to the drawings.

By applying the present invention for detecting a nucleic acid in an unknown sample, for verifying the quality of a produced microarray or for determining the concentration of the nucleic acid in the sample as demonstrated in each of the embodiments below, novel detection and assay processes are provided which saves time and labor compared to conventional processes.

### Embodiment 1

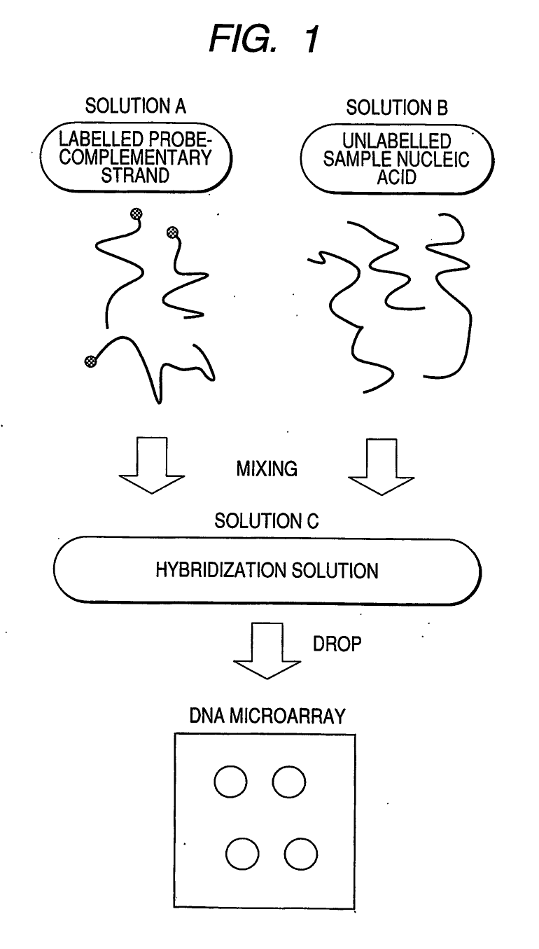

FIG. 1 illustrates steps in the assay process of the present invention. Solution A contains a labeled complementary nucleic acid that specifically binds to probe nucleic acids (hereinafter referred to as probe-complementary nucleic acid), and Solution B is a solution containing an unlabeled target nucleic acid derived from an unknown sample (hereinafter referred to a target nucleic acid or sample nucleic acid). Solution A and Solution B are mixed to prepare hybridization solution C. By spotting the solution C onto a DNA microarray, hybridization reaction occurs between the probe nucleic acids on the DNA microarray and a labeled probe-complementary nucleic acid or an unlabeled sample nucleic acid. For a labeled probe-complementary nucleic acid, a single-stranded nucleic acid having a sequence that hybridizes to a probe nucleic acid is preferably used.

One example of the probe nucleic acids immobilized on the substrate is an oligonucleotide having a base sequence capable of hybridizing to a target nucleic acid and a bonding part to the substrate.

The probe used for the probe array of the present invention is suitably selected according to the purpose, but it is preferably selected from the group consisting of DNA, RNA, cDNA (complementary DNA), PNA, oligonucleotide, polynucleotide and other nucleic acid for appropriate practice of the present invention, and if needed, two or more types of these can be used in combination.

The probe nucleic acids immobilized on the substrate can be prepared by the ink jet process etc. For example, a process as disclosed in the Japanese Patent Application Laid-Open No. H11-187900 can be applied.

The composition of the hybridization solution containing nucleic acid is not limited as long as desired hybridization reaction may occur. Any hybridization solution conventionally used in the art can be used.

When an oligonucleotide is used as a labeled probe-complementary nucleic acid to be contained in Solution A, the length of the oligonucletide is 100-mer or less. For example, the length may be the same as the probe nucleic acid.

The labeled oligonucleotide can be prepared with one-base extension by chemical synthesis, and the obtained oligonucleotide can be purified to almost 100% purity using techniques such as liquid chromatography. Therefore, n molecules of labeling compound can be precisely attached to one probe-complementary nucleic acid (n is a predetermined number and usually n=1), and such a labeled nucleic acid of high purity can be used for Solution A.

Accordingly, the quantity (number) of the labeled probe-complementary nucleic acid bound to the probe nucleic acid on the substrate under a condition where a sample nucleic acid is not present can be determined precisely by measuring the corresponding amount of the immobilized label on the array.

On the other hand, the conventional process for adding a label molecule to a nucleic acid derived from a living organism completely differs from this process. More specifically, one of the most commonly used processes for attaching a label molecule to the nucleic acid derived from a living organism is to use a labeled nucleotide as a substrate for enzyme reactions such as PCR. In this case, however, the probability that the enzyme binds a nucleotide to which a labeling molecule is attached is extremely low compared with the probability that enzyme binds an unlabeled normal nucleotide. Therefore, it is very difficult to introduce labeled molecules into the nucleic acid with sufficient reproducibility, and this has been a major cause for experimental error.

According to the present invention, the strength of hybridization is measured using a nucleic acid that has n labeling molecules per nucleic acid molecule without fail, therefore assay can be performed with high reproducibility. Details such as PCR reaction process, method and conditions for hybridization, process for detecting the labeled substance used in the present invention will be described in Examples.

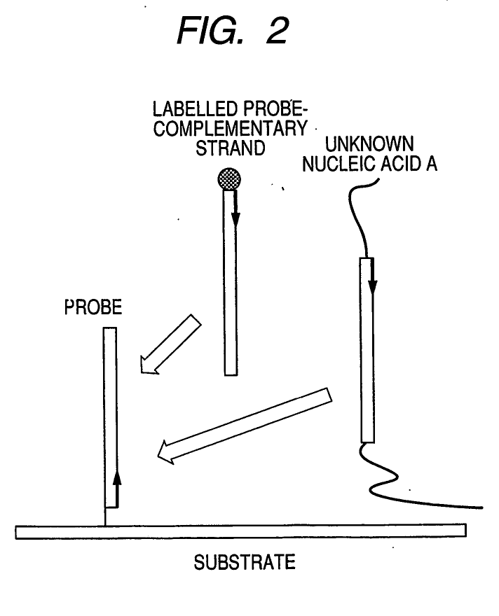

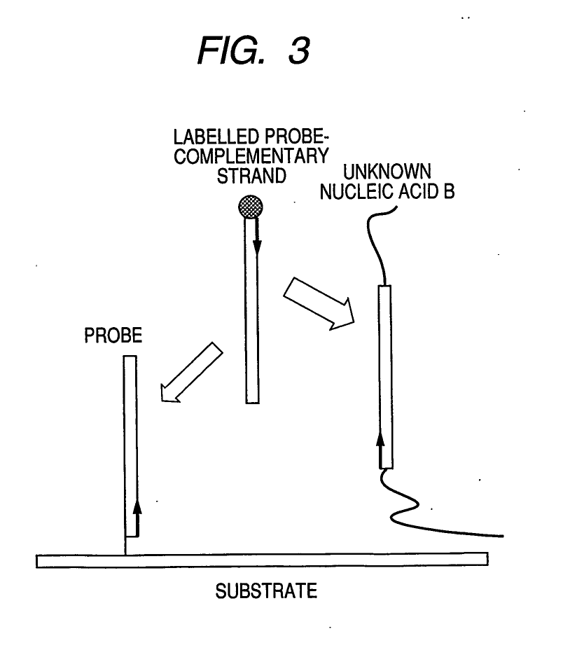

FIGS. 2 and 3 schematically show the reaction of the present invention. In these drawings, an immobilized probe nucleic acid is expressed as a bar attached to the substrate. It is essential that the base sequence of the probe nucleic acid is known. It may be either a cDNA or an oligonucleotide. In FIGS. 2 and 3, a labeled probe-complementary nucleic acid, which can specifically bind the probe nucleic acid and is contained in Solution A of FIG. 1, is represented by a bar with a black circle.

In FIG. 2, “unknown nucleic acid A” or “sample nucleic acid A” is a nucleic acid that is expected to hybridize with the probe nucleic acid, and is a single-stranded nucleic acid which will compete with the labeled probe-complementary nucleic acid. The sample nucleic acid may be RNA, single-stranded cDNA synthesized from RNA, DNA synthesized by asymmetrical PCR, etc.

The black arrow in the nucleic acids shows so-called from 5′-end to 3-end′ direction of the nucleic acid. In FIG. 2, the hybridization reaction shown by two white arrows will compete each other.

In FIG. 3, the unknown sample nucleic acid B is expected to hybridize with the labeled probe-complementary nucleic acid. The hybridization reactions shown by two white arrows will compete.

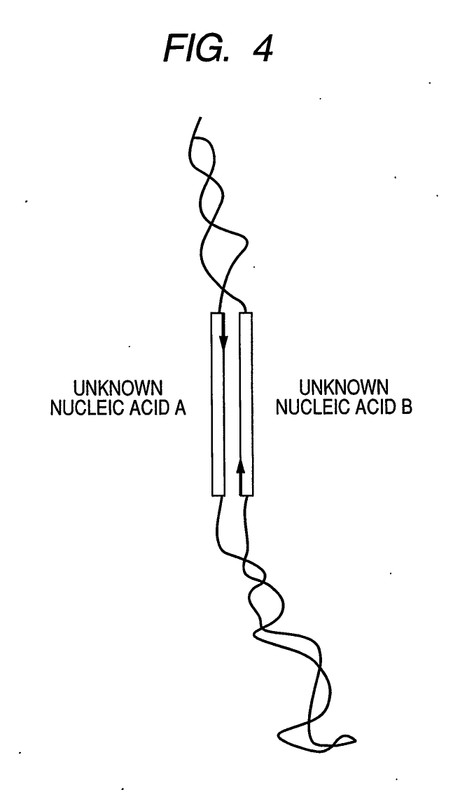

When the target nucleic acid A in FIG. 2 and the nucleic acid B in FIG. 3 are complementary with each other, their relation is as shown in FIG. 4.

For example, when ordinary PCR is performed with a sample, a double-stranded nucleic acid pair as shown in FIG. 4 will be contained in Solution B of FIG. 1. The amount of the labeling substance bound to the immobilized probe without any competition can be estimated when a nucleic acid having a very low possibility of being contained in the sample nucleic acid is immobilized as a positive control probe on the DNA microarray of FIG. 1 and Solution A further contains a labeled nucleic acid which will hybridize with the control probe specifically. In principle, the quantity of the target nucleic acid in the unknown sample can be estimated by comparing the amount of the label bound to the control probe with no competition and the amount of the label bound to the probe by hybridization of the probe-complementary nucleic acid contained in Solution C.

The concentration of the labeled probe-complementary nucleic acid in the hybridization solution should be adjusted in such a manner that the amount of the bound label will decrease when competition is present in comparison with the case of no competition.

Alternatively, an experimental process without positive control can be established by determining beforehand the standard values for the concentration of the labeled complementary nucleic acid in Solution A and the binding amount of the label to the probe to know decrease in the amount of the label bound to the probe from the standard value.

As described above, the assay process of the present invention estimates the amount of the target nucleic acid utilizing the fact that when a mixture of the labeled probe-complementary nucleic acid and the target nucleic acid is applied to an immobilized probe, the amount of the labeled complementary nucleic acid captured by the immobilized probe will decrease owing to competition with the target nucleic acid in comparison with the case where the hybridization solution contains the labeled probe-complementary nucleic acid but not the target nucleic acid.

### Embodiment 2

In order to carry out the quantification of the target nucleic acid from the unknown sample contained in Solution B in FIG. 1, this embodiment shows an assay process using dilution series.

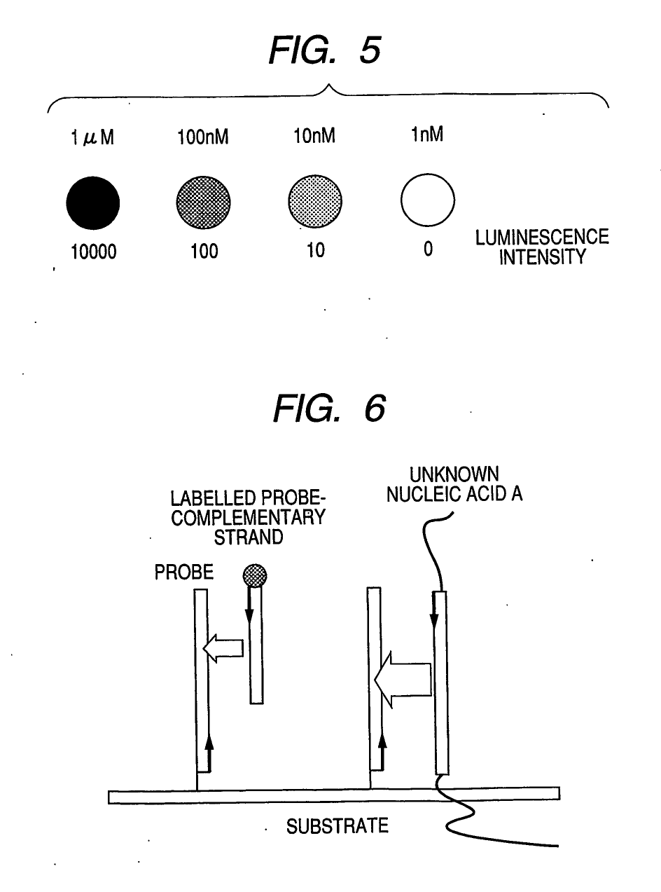

FIG. 5 schematically shows a hybridization result when the concentration of the labeled probe-complementary nucleic acid in Solution A of FIG. 1 was varied. FIG. 5 means that in the range of from 1 μM (μmol/1) to 100 nM, the concentration of the labeled probe-complementary nucleic acid is so high that no decrease is observed in the detected (remained) amount of the label after hybridization reaction with the probe explained in the embodiment 1. On the other hand, when the concentration of the complementary nucleic acid is lowered to 1 nM, the competitive hybridization reaction described in Embodiment 1 occurs, and the intensity of the detected (or remained) label becomes much lower than that observed when the hybridization solution contains only the labeled probe-complementary nucleic acid not the target nucleic acid. The examples of the measured intensity are also shown in FIG. 5. Consequently, the concentration of the target nucleic acid from a sample can be assumed to be between 1 nM and 100 nM. Based on this result, use of a still finer dilution series enables to estimate the concentration of the target nucleic acid more precisely.

Although in the above embodiment, the concentration of the target nucleic acid in Solution B of FIG. 1 is fixed while the concentration of the probe-complementary nucleic acid in Solution A is varied, the opposite is also possible, that is, the concentration of the labeled probe-complementary nucleic acid in Solution A is fixed and the concentration of the target nucleic acid in Solution B is varied.

### Embodiment 3

The nucleic acid containing a labeled complementary nucleic acid in Solution A of FIG. 1 exists as a molecule with very high purity as described in Embodiment 1. On the other hand, the nucleic acid from the sample in Solution B is synthesized through several steps of biochemical reactions, and there is a possibility of contamination of impurities in the synthetic process, and there may be nucleic acid of different lengths and types.

For this reason, the purity of the labeled complementary nucleic acid in the solution A is higher than that of the sample nucleic acid, so that, in principle, the hybridization with the labeled complementary nucleic acid in the solution A is stronger. Therefore, for the present invention where the quantity of the sample nucleic acid is estimated on the basis of reduction of the bound amount of the label, it may be necessary to make the quantity of the sample nucleic acid sufficiently large. In order to solve this problem, this embodiment describes how to detect target nucleic acid of a rather small amount.

As shown in FIG. 6, the length of the labeled probe-complementary nucleic acid in the solution A is made shorter than the probe, for example, the probe nucleic acid is 20-mer, and the labeled probe-complementary nucleic acid is 10-mer. As a result, the hybridization reaction between the probe and the complementary nucleic acid becomes weak. On the other hand, the target nucleic acid A in the solution B is usually much longer than 20-mer, and contains a portion entirely complementary to the probe nucleic acid. Therefore, in FIG. 6, the hybridization indicated by the white arrow (right) between the probe and the target nucleic acid A is stable compared with the hybridization indicated by the white arrow (left) between the probe and the labeled probe-complementary nucleic acid. As a result, one can observe decrease in the detected (or remained) amount of the label as illustrated in FIG. 5 even when the concentration of the target nucleic acid A is rather low.

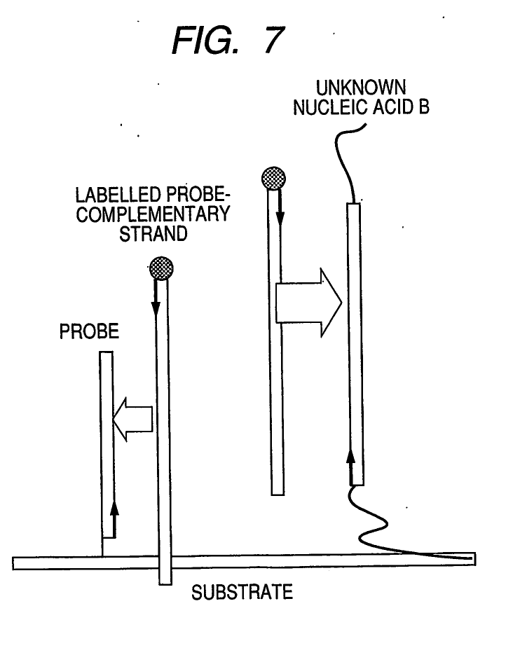

FIG. 7 shows an example in which the labeled probe-complementary nucleic acid in Solution A is made longer than the probe in the DNA microarray. For example, the length of the probes is set to 20-mer and the labeled probe-complementary nucleic acid is set to 30-mer. In this case, if the sequence of the extended portion is one expected to hybridize with the sample nucleic acid B, hybridization reaction between the labeled probe-complementary nucleic acid and the sample nucleic acid B becomes stronger. On the other hand, the length of the hybridizable portions of the probe and the labeled probe-complementary nucleic acid is still 20-mer. Therefore, as shown in FIG. 7, the hybridization reaction indicated by the right white arrow between the labeled probe-complementary nucleic acid and the unknown nucleic acid B becomes stronger than the hybridization reaction between the probe and the labeled probe-complementary nucleic acid indicated by the left white arrow, and the equilibrium shifts toward the release of the labeled probe-complementary nucleic acid increasing the chance in which the target nucleic acid A binds to the microarray. As a result, one can observe decrease in the detected (or remained) amount of the label as illustrated in FIG. 5 even when the concentration of the target nucleic acid A is rather low.

As shown above, by making the labeled complementary nucleic acid which specifically binds to a probe nucleic acid in a DNA microarray longer or shorter than the probe, decrease in the detected (or remaining) amount of the label bound to the probe can be observed even in when the concentration of the target nucleic acid is rather low.

### Embodiment 4

The process for analyzing nucleic acid concentration in this embodiment comprises the steps of introducing a solution containing a nucleic acid derived from an unknown sample into a chamber in which immobilized probe nucleic acids have been arranged; introducing a solution which contains a labeled probe-complementary nucleic acid that specifically binds to one of the immobilized probe nucleic acids, in such a manner that two solution are mixed; detecting the amount of the label bound to the immobilized probe; where the concentration of the nucleic acid derived from the unknown sample in the mixed solution is estimated based on the correlation between the introduced amount of the labeled probe-complementary nucleic acid into the above-described chamber and the change in the amount of the label attached to the immobilized probe due to the introduction. The detection of the amount of the label is suitably conducted using a confocal microscope.

The system for realizing this process and detailed assay process will be described below.

(Construction of the System)

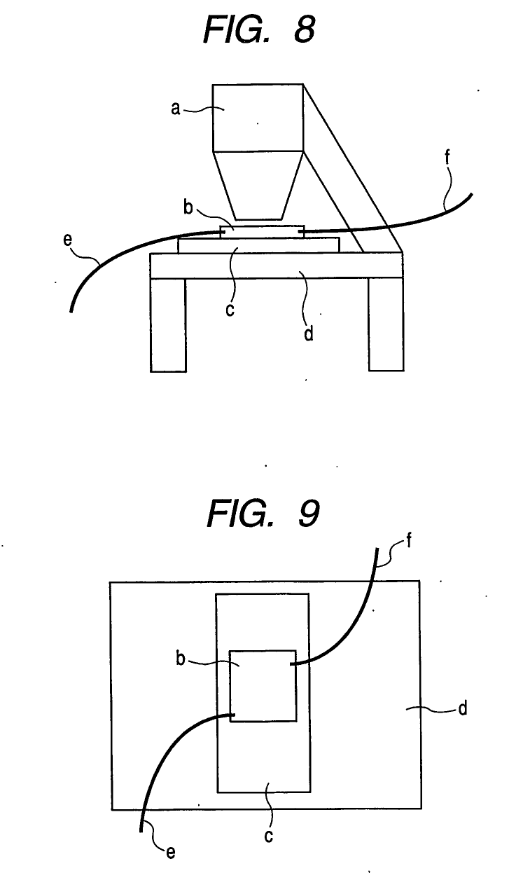

This whole image of this embodiment is shown in FIG. 8. A hybridization chamber (b) is fixed on the array (c) so as to cover the region where the probes are immobilized, and is sealed so that the solution may not leak. They are arranged so that the reaction of the probe and labeled probe-complementary nucleic acid can be observed under the confocal microscope.

As shown in FIGS. 8, 9, the microarray and the hybridization chamber are installed on the stage (d) which temperature control is possible, and the hybridization chamber is equipped with inlet pipe (f) and discharge pipe (e) at the upper right and lower left portion of the chamber respectively. The hybridization solution and washing solution are introduced in and are discharged out of the chamber through these pipes.

The laser used for confocal microscope is suitably chosen according to the label used. Helium neon laser of a wavelength of 543 nm is suitable for observation of a fluorescence coloring substance such as rhodamine. The microscope is adjusted to focus on the surface of the microarray. If such an adjustment is performed, the fluorescence only from the labeled probe-complementary nucleic acid hybridized with the probe can be observed as a spot in spite of the noise from the fluorescent substance present in the solution. Hybridization of each probe can be evaluated from the intensity of this fluorescence.

(Operation of the System)

Analyzing process using the system set as mentioned above specifically is shown below.

A hybridization solution shown in Embodiment 1 is introduced from the inlet pipe (f). While the temperature is controlled by temperature controller, the focus of the confocal microscope is adjusted to be on the substrate surface either automatically or manually.

A marker probe that emits fluorescence even when it is not hybridizing with the labeled probe-complementary nucleic acid may be provided on the microarray. Such a marker probe usable here may be fluorescence coloring substances having thiol as a functional group, the details thereof are disclosed in Japanese Patent Application Laid-Open No. H07-27768, etc. The focus of the microscope may be adjusted using the fluorescence from the marker probe as a guide.

Then, a solution containing only the labeled probe-complementary nucleic acid is introduced from the inlet pipe (f), with a predetermined temperature maintained, to change the concentration of probe complementary nucleic acid in the hybridization chamber. Light agitation is applied to the chamber so that the concentration of the introduced labeled probe-complementary nucleic acid becomes uniform in the chamber. The solution of the labeled probe-complementary nucleic acid is prepared at a higher concentration and the solution is introduced little by little so that the concentration of the target nucleic acid from the unknown sample in the hybridization chamber will not change much upon introduction of the labeled probe-complementary nucleic acid.

The fluorescence signal from the spot is confocally observed and when the spot begins to be observed, the introduction rate of the solution containing the labeled probe-complementary nucleic acid is preferably further decreased. The above-described solution is introduced in such a manner that an equilibrium state may be maintained as much as possible, and a correlation between the concentration of the labeled complementary nucleic acid in the hybridization chamber and the fluorescence intensity is obtained, thereby enabling more precise concentration assay of unknown samples.

That is, in this embodiment, the state in which fluorescence is emitted from the spot portion of the probe on the substrate can be observed simultaneously with the hybridization reaction between the probe and the complementary nucleic acid using a confocal type microscope. By controlling the introduced amount of the complementary nucleic acid based on the result of this fluorescence observation, the concentration of the unlabeled nucleic acid derived from the unknown sample and that of the labeled probe-complementary nucleic acid in the hybridization chamber can be adjusted to the same level, and thereby more exact concentration of the unlabeled target nucleic acid derived from the unknown sample can be estimated.

### Embodiment 5

In this embodiment, the treating process of the present invention is applied for verifying the quality of the manufactured DNA microarray.

The conventional processes have a problem that whether the desired probe DNA is present on the DNA microarray substrate or not cannot be known when it is manufactured, since the DNA itself has no fluorescence properties.

It is not impossible to check all DNA arrays manufactured by using an expensive assay method to verify the existence of DNA, but it is very costly and therefore practically not applicable. That is, in the conventional processes, quality evaluation of the DNA microarray on “whether all the probe spots exist at the exact positions” is only achieved by random inspection before the microarrays are subjected to hybridization reaction with a solution containing labeled sample nucleic acid for detection.

Moreover, the quantity of the DNA molecules varies in each probe spot on the DNA microarray. Conventionally, this variation is eliminated as much as possible in the manufacturing process of DNA microarray and the subsequent experiment is conducted based on the assumption that “DNA molecules are present in every probe spot in the same amount.” However, often this assumption is not the case. If the experiment can be conducted after the actual amount of immobilized probe DNA molecules has been determined, the accuracy of the experiments will rise. It was practically impossible, however, to measure the amount of the attached probe DNA before the experiment by the conventional process as above mentioned.

Moreover, introducing a label to the nucleic acid in an unknown sample is a cost- and time-consuming laborious task and the rate of introducing a label into the sample nucleic acid is often variable, causing a problem that the quantitativity of the experiment system using the DNA microarray is hard to be guaranteed.

This embodiment solves the above-described problems.

The detection process of the present invention is also applicable to a system to detect difference in genome, so-called SNP (Single Nucleotide Polymorphism) using a DNA microarray.

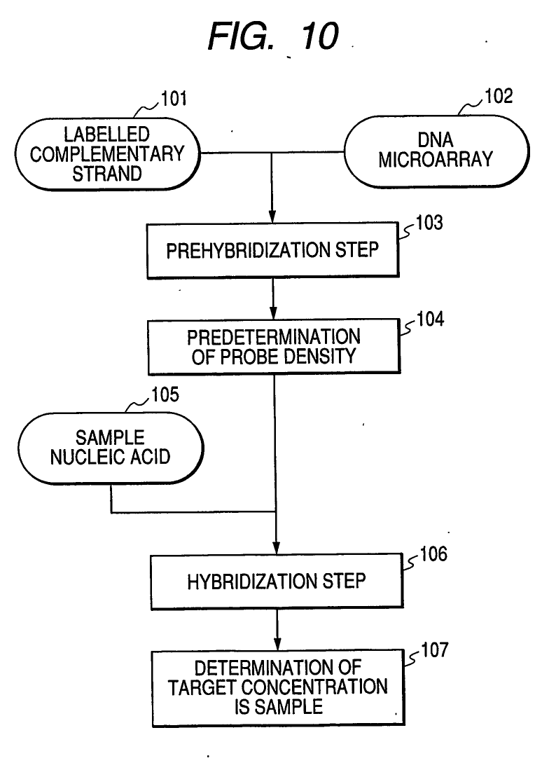

FIG. 10 best illustrates the assay process of this embodiment: illustrating each steps in the DNA microarray assay process of the present invention.

Numeral 101 represents a labeled probe-complementary nucleic acid taht can specifically bind to the probe nucleic acid. An oligonucleotide of 100 or less nucleotides is preferably used in the present invention.

The labeled oligonucleotide can be prepared via one-base extension by chemical synthesis, and purified to almost 100% purity using a technique such as liquid chromatography. Therefore, labeled probe-complementary nucleic acid can be precisely labeled with n (usually one) labeling molecules per oligonucleotide molecule.

In the prehybridization step 103, this labeled molecule is applied to the DNA microarray 102 for hybridization. Usually, a washing step is also included in this prehybridization step 102. In order to stabilize the quality to ensure reproducibility in the evaluation using the microarray, it is important to surely make immobilized probes in a hybrid state at this process.

Then the quantity of the labeled complementary nucleic acid 101 fixed at the probe site after prehybridization is determined. This is the step 104 for predetermining probe density. In this step 104, the hybridization intensity is measured to determine the density of the bound probe 101 using a probe-complementary nucleic acid which has been precisely labeled with n labeling molecules per molecule as described above. Hence experiments can be performed with very high reproducibility.

At present, the DNA microarray is used on an assumption that DNA molecules are present in every probe spot in the same amount. However, it is often rational to consider that the amount of the probe nucleic acid varies spot to spot. It is practically impossible, however, to measure the amount of the attached probe DNA before the experiment by the conventional processes, so that the subsequent experiment has been conducted on that assumption. On the other hand, according to the assay process of the present invention, variation in the amount of the immobilized nucleic acid can be measured precisely, subsequent experiments can determine more precisely.

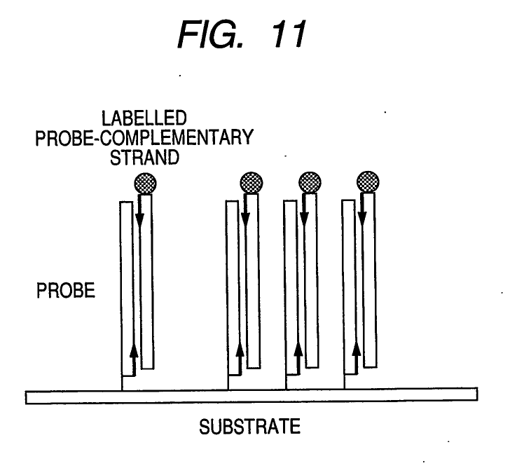

The state of the DNA microarray at this point is illustrated in FIG. 11. In FIG. 11, the probe DNA of the DNA microarray of the present invention is expressed as a bar attached to the substrate, where, as a result of hybridization reaction with an excessive amount of the labeled probe-complementary nucleic acid (101), almost all the probe DNA molecules are bound to labeled complementary nucleic acid 101.

In this point, preferably 60% or more, more preferably 80% or more, still more preferably 90% or more of the probe nucleic acid immobilized on the substrate are bound to the labeled complementary nucleic acid.

Numeral 105 represents a nucleic acid extracted from an unknown sample and optionally amplified (sample nucleic acid). The detailed process will be described later. Numeral 106 represents a hybridization step, where hybridization is carried out between the DNA microarray in the state shown in FIG. 11 and the sample nucleic acid. Usually, the hybridization step 106 includes a washing process. In conventional hybridization reaction, a fluorescence coloring substance or a reactive molecule such as biotin that can react with the labeling substance is introduced to the sample nucleic acid 105. According to the present invention, the labeled complementary nucleic acid 101 is bound to the DNA microarray in the step 104 of the predetermination of probe density as shown in FIG. 11. Thus, if necessary, another type of labeling substance different from the label attached to the complementary nucleic acid 101 may be introduced into the sample nucleic acid 105 for better results. If necessary, the labeled complementary nucleic acids attached to the probes as shown in FIG. 11 can be removed after the predetermination of probe density step 104, for example, by washing DNA microarray at a high temperature of 90° C. or more may be inserted.

Finally in the determination step 107, the exact quantification of the sample nucleic acid (105) derived from the unknown sample can be made by measuring the quantity of the sample nucleic acid (105) bound to the DNA microarray, with calibration using the result of the predetermination probe density step 104.

### Embodiment 6

In this embodiment, a nucleic acid assay process for detection of gene polymorphism is explained in detail.

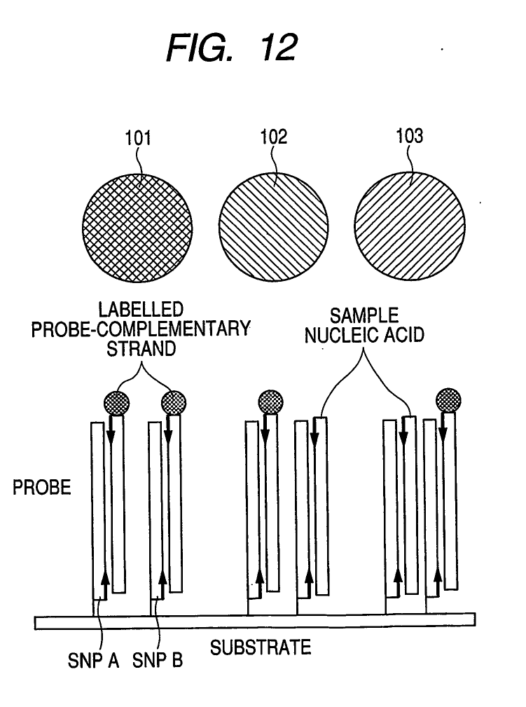

FIG. 12 shows steps in the assay process for this embodiment. The large circles on the upper part of the FIG. 12 schematically illustrate the state of probe spots on the DNA microarray, here, three states 101 to 103.

The drawing under the circles schematically illustrates probe DNAs and nucleic acids hybridized thereto in respective spots, which are represented by two adjacent bars. Although there are many probes in one spot, only two are shown in FIG. 12. In addition, the arrow written in the bar shows the 5′-3′ direction of the nucleic acid.

In the DNA microarray of FIG, 12, two kinds of probes corresponding to two alleles of SNP are immobilized on the same spot. Usually, SNPs are one-point (base) polymorphism, and rarely have three or more alleles, but the present invention can be applied also to SNPs of three or four alleles, and can be also applied to gene polymorphism other than SNPs.

The probe nucleic acid of “SNP A” in the spot of FIG. 12, is assumed to have a sequence, for example, ATCGGGATTAGCGATTCAGTA and the probe nucleic acid of “SNP B” ATCGGGATTACCGATTCAGTA. This means that the base at the central position of the probe sequence is “G” for allele A and “C” for allele B. In this case, the labeled probe-complementary nucleic acid is “TACTGAATCGCTAATCCCGAT” for allele A and “TACTGAATCGGTAATCCCGAT” for allele B. The small circle attached on the bar representing the labeled probe-complementary nucleic acid is a labeling substance. The labeling substances of different properties are used for respective alleles. A fluorescent labeling substance is commonly used recently, and the present invention can be carried out by using substances different in fluorescence emission wavelength, for example, Cy3 for red fluorescence and Cy5 for green fluorescence.

The spot 101 shows the state where the labeled probe-complementary nucleic acids having completed hybridization reaction with allele A/B respectively as is shown at the lower part of FIG. 12. For example, when Cy3 is attached to the probe complementary nucleic acid for allele A as a labeling substance to show red color and Cy5 is attached to the probe complementary nucleic acid of allele B to show green color, the spot 101 where these two labeled probe-complementary nucleic acids hybridized to alleles A and B respectively, the color of spot 101 becomes yellow, a mixture of these two colors. It should be noted, however, these colors are only for explanation and not limited thereto.

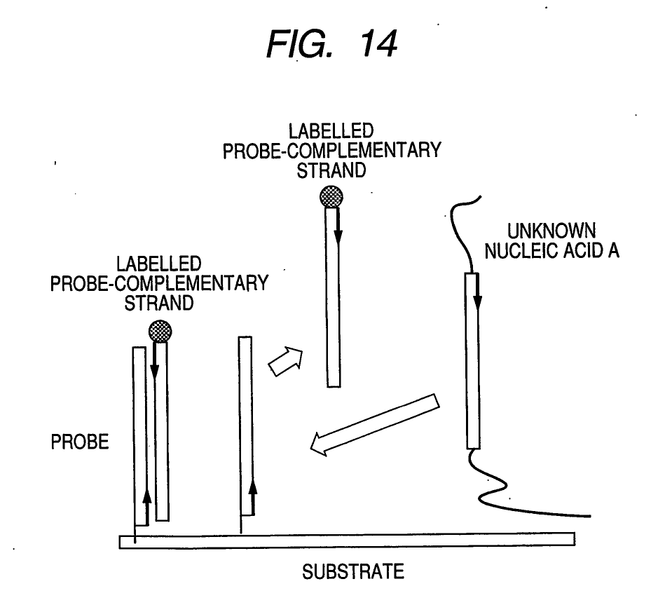

While two kinds of labeled probe-complementary nucleic acids have hybridized to the probes in mostly the same amount in the spot 101, in spot 102 or spot 103, part of the labeled probe-complementary nucleic acid is replaced by a nucleic acid from the sample causing deviation in the fluorescent substances. In the spot 102, the labeled complementary nucleic acid for allele B is replaced with the sample nucleic acid. Since no labeling substances has not been introduced into this sample nucleic acid, the fluorescence of the spot 102 is more from Cy3. Schematically, it will shift toward pure red from the yellow in which two colors were mixed. Contrary, in the spot 103, the labeled complementary nucleic acid for allele A is replaced with the nucleic acid from the sample. The fluorescence from the spot 103 comes more from Cy5. Saying schematically, it will shift toward pure green from yellow of mixed two colors. Thus from the color shift from a color of equal mixture, one can know which allele is contained in the sample nucleic acid. The principle of this substitution is explained in detail using FIG. 14 and subsequent drawings.

Although here is shown an example where all the alleles of polymorphism are immobilized in one spot, probes for each allele may be immobilized in different spots without changing the essence of the present invention. For example, only SNP A may be immobilized in spot 101 as a probe, and only SNP B on spot 102.

However, in the case of polymorphism consisting of only two alleles like SNPs, immobilizing two alleles in one spot as in FIG. 12 has an advantage that analysis of the result becomes easy.

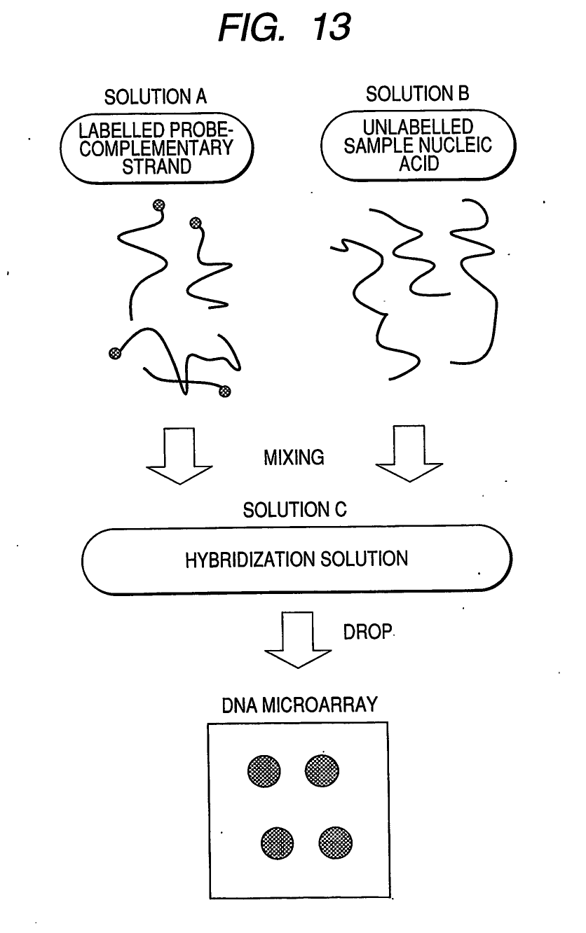

FIG. 13 shows the assay process of the present invention. Solution A contains the labeled probe-complementary nucleic acid that specifically binds to a probe nucleic acid, and Solution B contains an unlabeled target nucleic acid from a sample solution A and Solution B are mixed to prepare solution C as a hybridization solution. By spotting the solution C onto a DNA microarray, the probe nucleic acid on the DNA microarray and a labeled probe-complementary nucleic acid or an unlabeled target nucleic acid are subjected to hybridization reaction. For a labeled probe-complementary nucleic acid, a single-stranded nucleic acid having a sequence that hybridizes to a probe nucleic acid is preferably used. The nucleic acid from the unknown sample is usually amplified using techniques such as PCR.

The composition of the hybridization solution containing nucleic acid is not limited as long as desired hybridization reaction may occur. Any hybridization solution conventionally used in the art can be used.

When an oligonucleotide is used as a labeled probe-complementary nucleic acid to be contained in Solution A, the length of the oligonucletide is 100-mer or less. For example, the length may be the same as the probe nucleic acid. The labeled oligonucleotide can be prepared with one-base extension by chemical synthesis, and the obtained oligonucleotide can be purified to almost 100% purity using techniques such as liquid chromatography. Therefore, n molecules of labeling compound can be precisely attached to one probe-complementary nucleic acid (n is a predetermined number and usually n=1), and such a labeled nucleic acid of high purity can be used for Solution A. The solution A contains at least two kinds of labeled probe-complementary nucleic acid corresponding to at least two alleles. In FIG. 13, two kinds of labeling substances (for example, Cy3 and Cy5) are attached to the probe-complementary nucleic acids respectively.

On the other hand, the conventional process for adding a label molecule to a nucleic acid derived from a living organism completely differs from this process. More specifically, one of the most commonly used processes for attaching a label molecule to the nucleic acid derived from a living organism is to use a labeled nucleotide as a substrate for enzyme reactions such as PCR. In this case, however, the probability that the enzyme binds a nucleotide to which a labeling molecule is attached is extremely low compared with the probability that enzyme binds an unlabeled normal nucleotide. Therefore, it is very difficult to introduce labeled molecules into the nucleic acid with sufficient reproducibility, and this has been a major cause for experimental error.

According to the present invention, the strength of hybridization is measured using a nucleic acid that has n labeling molecules per nucleic acid molecule without fail, therefore assay can be performed with high reproducibility.

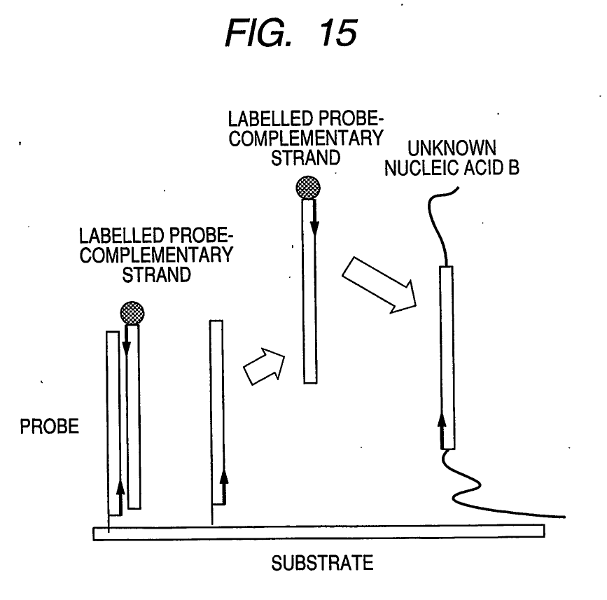

FIGS. 14 and 15 schematically show the reaction of the present invention. In these drawings, an immobilized probe nucleic acid is expressed as a bar attached to the substrate. It is essential that the base sequence of the probe nucleic acid is known. It may be either a cDNA or an oligonucleotide. In FIGS. 14 and 15, a labeled probe-complementary nucleic acid, which can specifically bind the probe nucleic acid and is contained in Solution A of FIG. 13, is represented by a bar with a black circle.

In FIG. 14, “unknown nucleic acid A” or “sample nucleic acid A” is a nucleic acid that is expected to hybridize with the probe nucleic acid, and is a single-stranded nucleic acid which will compete with the labeled probe-complementary nucleic acid. The sample nucleic acid may be RNA, single-stranded cDNA synthesized from RNA, DNA synthesized by asymmetrical PCR, etc.

The black arrow in the nucleic acids shows so-called 5′to 3′ direction of the nucleic acid. In FIG. 14, the hybridization reaction shown by two white arrows will compete each other.

When a competitive reaction as shown in FIG. 14 occurs, the shift of the detected concentration (remaining concentration) of the label is observed. For example, without competition, both of the two kinds of labeled probe-complementary nucleic acids hybridize as in spot 101 and two kinds of fluorescence is mixed and detected as shown in FIG. 12. On the other hand, when the allele portion of the nucleic acid A in an unknown sample hybridizes to some of the probes in FIG. 14, which causes a shift to either one of the fluorescences as in the spots 102 or 103. Thus, the allele of the nucleic acid in a sample can be determined in principle by measuring shift of the fluorescence.

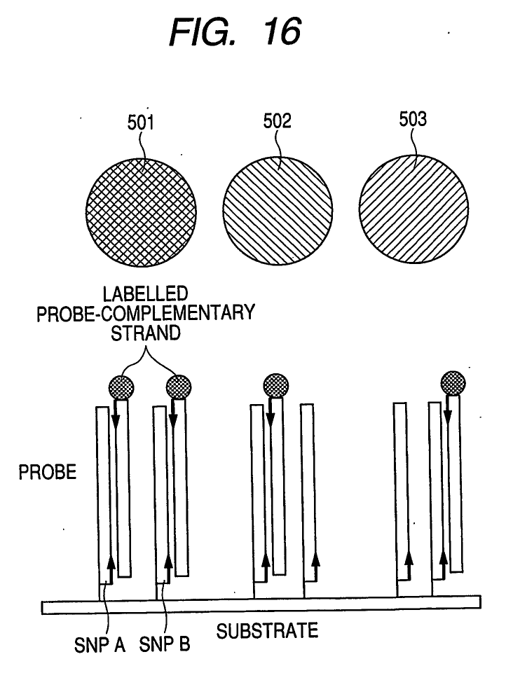

As shown in FIG. 15, if the unknown nucleic acid B expected to hybridize to one of the labeled probe-complementary nucleic acids is present, two hybridization reactions shown by two white arrows will compete. When a competitive reaction as shown in FIG. 15 occurs, a shift of the detected concentration (remaining concentration) of the label is observed. For example, referring to FIG. 16, while all of the two kinds of labeled probe-complementary nucleic acids will originally cause hybridization reaction like 501 and two kinds of fluorescence should be mixed and detected, the nucleic acid B will hybridize to some of the labeled probe-complementary nucleic acid to decrease the quantity of the labeled probe-complementary nucleic acid bound to generate free probes not hybridizing with the labeled complementary nucleic acid. Consequently, shift from one fluorescence to another as in the spot 502 or 503. Thus the nucleic acid in the sample can be determined to be either one of the alleles by measuring fluorescence shift.

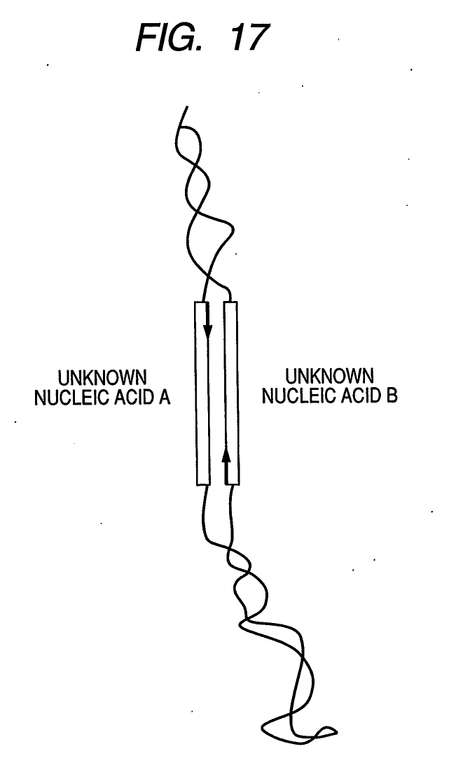

The relation between the nucleic acid A of FIG. 14 and the nucleic acid B of FIG. 15 is shown in FIG. 17. When the usual PCR or the like is performed for a sample, the nucleic acid pair shown in FIG. 17 becomes a main component of Solution B of FIG. 13. At this time, the competitive reactions of FIG. 14 and FIG. 15 will occur simultaneously.

If the condition of the spot where no competitive reaction occurs as shown in FIG. 14 or FIG. 15 will occur, that is, two kinds of labeling molecules are present in the same amount in the spot, is known beforehand, allele type of the unknown sample will be known by detecting the shift from such a state. When the state of the spot without competitive reaction like 101 of FIG. 12 or the spot of 501 of FIG. 16 is not known beforehand, the balance of the amount of the labeling substances bound to the immobilized probe without any competition can be estimated by immobilizing two kinds of nucleic acids having a very low possibility of being contained in the sample nucleic acid as positive control probes on the DNA microarray, and by adding to Solution A further labeled nucleic acids which will hybridize with the control probes specifically. In principle, the quantity of the target nucleic acid in the unknown sample can be estimated by observing the shift from the balanced condition.

The concentration of the labeled probe-complementary nucleic acid in the hybridization solution should be adjusted in such a manner that the amount of the bound label will decrease when competition is present in comparison with the case of no competition.

### Embodiment 7

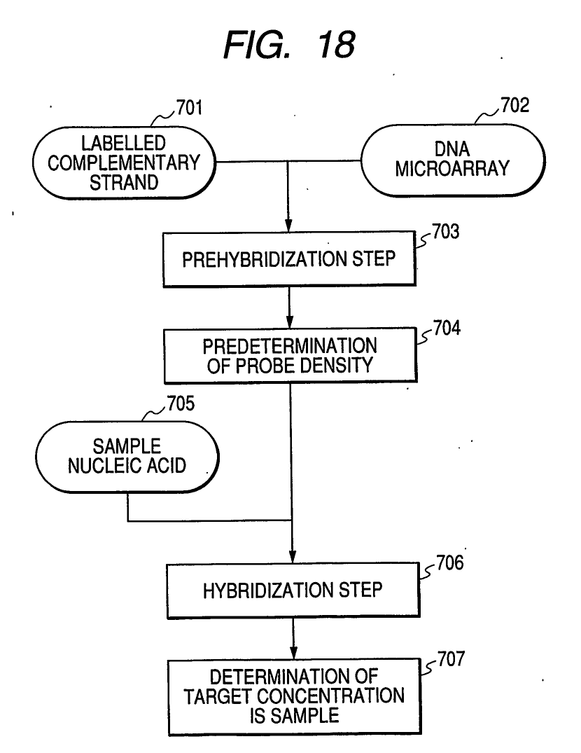

In the above embodiment 6, the labeled probe-complementary nucleic acids shown in FIG. 12 are mixed with a nucleic acid from a sample before hybridization reaction. In this embodiment, two-stage reactions as shown in FIG. 18 is carried out, that is, the labeled probe-complementary nucleic acids are subjected to hybridization reaction with a DNA microarray first, and then the nucleic acid from the unknown sample is hybridized.

Numeral 701 represents labeled complementary nucleic acids that can specifically bind probe nucleic acids, and are equivalent to the labeled complementary nucleic acids being a main component of Solution A of FIG. 13.

At the prehybridization step 703, hybridization reaction between the labeled complementary nucleic acids 701 and the DNA microarray 702 is conducted. Usually, a washing step is also included in this prehybridization step 703. In order to bind the labeled probe-complementary nucleic acids to almost all the probes immobilized on the DNA microarray, the prehybridization step of 703 is typically performed using the labeled probe-complementary nucleic acids in an excessive amount.

The quantity of labeled complementary nucleic acids 701 which remain at probe positions as a result of prehybridization step is determined in the probe-density predetermination step 704. Since the degree of hybridization is measured using a probe nucleic acids (701) ensured to be precisely labeled with n labeling molecules (n is predetermined by labeling control, typically at n=1 for a single complementary nucleic acid) per molecule in this predetermination step, assay with extremely high reproducibility can be performed.

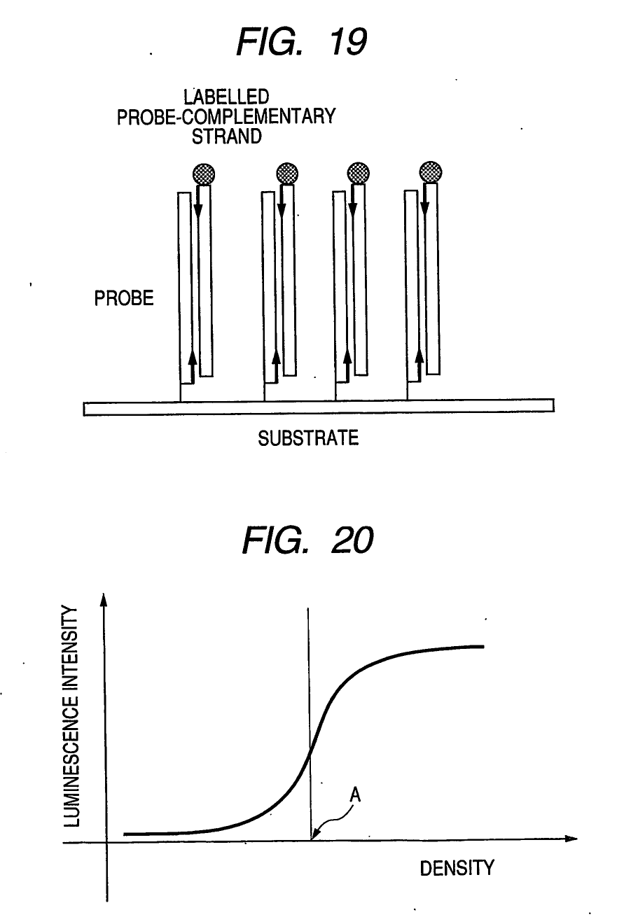

The state of the DNA microarray at this point is shown in FIG. 19 with bars attached to the substrate. Since sufficiently excessive amount of the labeled complementary nucleic acids (701) was used for hybridization reaction, almost all the probe DNA molecules are binding to the nucleic acids (701).

Numeral 705 is a nucleic acid extracted from the unknown sample and optionally amplified. Numeral 706 represents a hybridization step, where hybridization is carried out between the DNA microarray in the state shown in FIG. 19 and the sample nucleic acid. Usually, the hybridization step 706 includes a washing process. If necessary, the labeled complementary nucleic acids attached to the probes as shown in FIG. 19 can be removed after the predetermination step 704, for example, by washing DNA microarray at a high temperature.

Finally, as a result of the competitive reaction occurred in the hybridization step of 706 as described referring to FIG. 14 or FIG. 15, the shift of the remained labeling substances as seen in the spots 102 or 103 in FIG. 12 or the spots 502 or 503 in FIG. 16 will be observed. Measuring this shift at the polymorphism detecting step 707 and comparing with the result of the predetermination step 704, one can determine the allele of the nucleic acid from the unknown sample.

As described above, according to the assay process of the present invention, part of the complementary nucleic acids hybridized to the probe nucleic acid immobilized on the substrate is replaced with unknown nucleic acid by competition between the unknown nucleic acid and the labeled complementary nucleic acid to cause shift in the balance of the attached labeled nucleic acids, which is utilized to estimate the type of allele of the unknown nucleic acids.

### EXAMPLES

The detailed examples of each step in each embodiment are shown below. The following examples should be construed to be illustrative, and the present invention is not restricted to the following specific processes, a reagent, and a product.

### Example 1

An example amplification reaction (PCR) of the nucleic acid from a sample is shown below.

(PCR Reaction Liquid Composition)

| | | |
|---|---|---|
| Premix PCR Reagent (TAKARA ExTaq) | 25 | μl |
| Template Genome DNA | 2 | μl (100 ng) |
| Forward Primer mix | 2 | μl (20 pmol/tube) |
| Reverse Primer mix | 2 | μl (20 pmol/tube) |
| H₂O | 19 | μl |
| Total | 50 | μl |

The reaction mixture of the above composition is subjected to an amplification reaction using a thermal cycler according to the following temperature cycle protocol of:

25 cycles of 95° C.-10 minutes; 92° C.-45 seconds; 55° C.-45 seconds; and 72° C.-45 seconds as one cycle, and finally 72° C.-10 minutes.

After the reaction is completed, primers are removed by using a purification column (QIAGEN QIAquick PCR Purification Kit: product of QIAGEN), the quantification of the amplified product is performed. The PCR amplified product is dissolved in TE to be 3 ng/μl.

(Blocking of DNA Microarray)

Blocking of the DNA microarray is performed in order to prevent the nucleic acid molecules from adhering to the portions other than the probes of the DNA microarray. It is commonly carried out just before hybridization.

BSA (bovine serum albumin Fraction V: product of Sigma) is dissolved in a 100 mM NaCl/10 mM phosphate buffer to be an about 1 wt % solution and a DNA microarray is soaked in this solution at room temperature for 2 hours. After the blocking completed, the array was washed with a 2×SSC solution which contains 0.1 wt % SDS (sodium dodecyl sulfate) and then rinsed with pure water, and the water was removed by using a spin dryer.

(Hybridization)

The DNA microarray is then set in a hybridization apparatus (Genomic Solutions Inc. Hybridization Station) and hybridization reaction is performed using the following hybridization solution and the conditions. The reaction may be done manually using a glass slide and a hybridization chamber instead of a hybridization apparatus.

(Hybridization Solution)

An example composition of the hybridization solution (solution C of FIG. 1) is as follows:

6×SSPE/10% Formamide/Target (nucleic acid from an unknown sample, PCR product: 500 ng)/labeled probe-complementary nucleic acid (final concentration: 1 nM).

500 ng of the amplified unknown nucleic acid of a sample is dissolved in a buffer (SSPE), to which formamide is added to a final concentration of 10%, and a labeled probe-complementary nucleic acid is added to a final concentration of 1 nM. Thus a hybridization solution is prepared. The buffer concentration (SSPE) is calculated beforehand to be 6×SSPE in the final solution. The final amount of the solution is prepared preferably in a range between 20 μl and 200 μl.

After warmed at 65° C. and held for 3 minutes, the above hybridization system is further held at 92° C. for 2 minutes and then at 45° C. for 3 hours. Then, the array is washed with 2×SSC and 0.1% SDS at 25° C. The array is further washed with 2×SSC at 20° C., and if necessary rinsed with pure water according to the usual manual to remove labeled probe-complementary nucleic acid not reacted with the probe and water is drained off by a spin dryer.

(Labeling/Fluorescence Measurement)

Fluorescence measurement of the DNA microarray after the hybridization reaction is performed using a fluorescence detection apparatus for DNA microarrays (GenePix 4000B, product of Axon) under the following condition:

The wavelength for fluorescence measurement is adjusted to the emission wavelength of the fluorescent substance contained in the labeled probe-complementary nucleic acid, and the excitation light intensity was adjusted so that the measured fluorescence intensity is 30,000 or less.

In order that a user may readily carry out the above-described process, it is also suitable to prepare the immobilized probe nucleic acid and the solution containing the above-described labeled probe-complementary nucleic acid as a kit. In this case, it is also preferable to prepare a solution including every probe-complementary nucleic acid complementary to every nucleic acid immobilized as a probe.

### Example 2

PCR amplification of the nucleic acid from a sample and blocking of the DNA microarray is carried out in the same manner as in Example 1. 500 ng of the PCR product is dissolved in a buffer (SSPE) and formamide is added to 10% to prepare a hybridization solution.

(Adjustment of the System)

The total image of this example is shown in FIG. 8. A confocal microscope (a) (LSM510, product of Carl-Zeiss) is installed, and a microarray (c) is fixed to the focal portion. A hybridization chamber (b) is fixed on the array (c) so as to cover the region where the probes are immobilized, and is sealed so that the solution may not leak. The height of the chamber is adjusted to be less than the focal length of the confocal microscope, so that the reaction of the probe and labeled probe-complementary nucleic acid can be observed under the confocal microscope.

As shown in FIGS. 8, 9, the microarray and the hybridization chamber are installed on the stage (d) which temperature control is possible, and the hybridization chamber is equipped with inlet pipe (f) and discharge pipe (e) at the upper right and lower left portion of the chamber respectively. The hybridization solution and washing solution are introduced in and are discharged out of the chamber through these pipes.

The confocal microscope uses helium neon laser of a wavelength of 543 nm suitable for observation of a fluorescence coloring substance such as rhodamine, and it is adjusted to focus on the surface of the microarray. If such an adjustment is performed, the fluorescence only from the labeled probe-complementary nucleic acid hybridized with the probe can be observed as a spot in spite of the noise from the fluorescent substance present in the solution. Hybridization of each probe can be evaluated from the intensity of this fluorescence.

(Hybridization)

The DNA microarray from which water is drained off is mounted on the stand (d) of the hybridization apparatus shown in FIG. 8, and set so that the hybridization chamber (b) may come on the probe area. After introducing the hybridization solution prepared as above from the inlet pipe (f), it is warmed at 65° C. and held for 3 minutes, then held at 92° C. for 2 minutes and held at.45° C. In this state, the confocal microscope is adjusted to focus on the substrate surface.

On the microarray, a marker probe that emits fluorescence without hybridization with the labeled probe-complementary nucleic acid is provided. The marker probe here is a fluorescent coloring substance having thiol as a functional group, of which detail is disclosed in Japanese Patent Application Laid-Open No. H07-27768, etc. Here, tetramethylrodamin to which thiol has been introduced is used as a marker. The focus of the microscope is adjusted using the fluorescence of the marker probe as a guide.

Then, maintaining the temperature at 45° C., a hybridization solution shown below containing only the probe-complementary nucleic acid is slowly introduced from the inlet pipe (f) to change the concentration of probe-complementary nucleic acid in the hybridization chamber.

Hybridization Solution 2

6×SSPE/10% Formamide/labeled probe-complementary nucleic acid (3 μM)

Light agitation is applied to the chamber so that the concentration of the introduced labeled probe-complementary nucleic acid becomes uniform in the chamber. The solution of the labeled probe-complementary nucleic acid is prepared at a higher concentration and the solution is introduced little by little so that the concentration of the target nucleic acid from the unknown sample in the hybridization chamber will not change much upon introduction of the labeled probe-complementary nucleic acid.

The fluorescence signal from the spot is confocally observed and when the spot begins to be observed, the introduction rate of the solution containing the labeled probe-complementary nucleic acid is preferably further decreased. The above-described solution is introduced in such a manner that an equilibrium state may be maintained as much as possible, and a correlation between the concentration of the labeled complementary nucleic acid in the hybridization chamber and the fluorescence intensity is obtained.

The relation between the concentration of the labeled probe-complementary nucleic acid in the hybridization chamber and the fluorescence intensity from the spot is shown in FIG. 20. Actually, the unknown sample target is a long chain DNA (PCR product) and the probe complementary nucleic acid is an oligo DNA, and therefore hybridization efficiency is not the same. For this reason, the concentration at the half of the saturated fluorescence intensity, point A in FIG. 20, cannot be made the concentration of the unknown sample directly. However, since for such an oligo DNA and the PCR product, a calibration curve can be drawn beforehand of the concentration ratio and the fluorescence intensity for known concentrations, the unknown sample concentration can be estimated by comparison therewith.

### Example 3

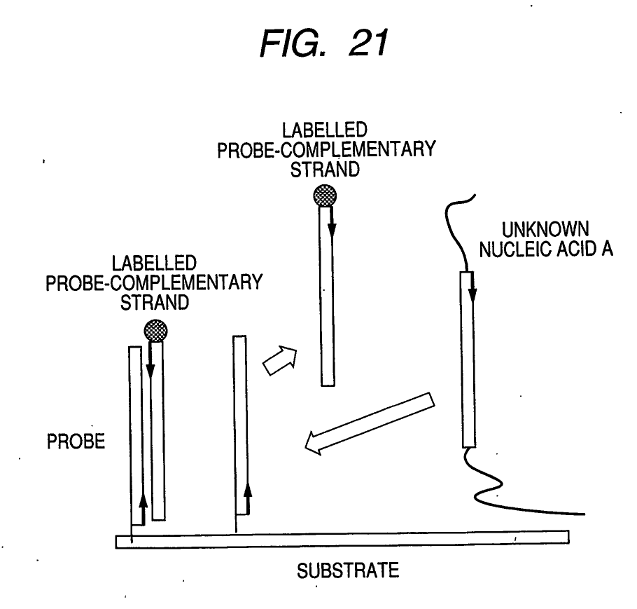

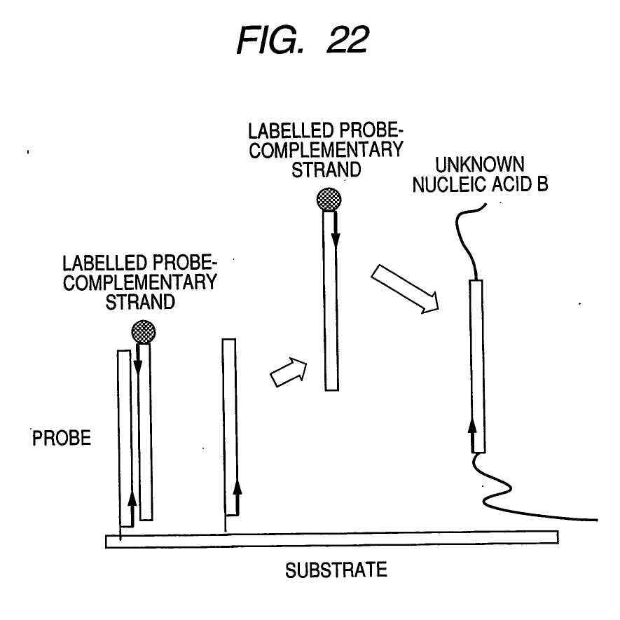

In Example 1, the labeling substance is mixed with the nucleic acid derived from the unknown sample (105). In this Example, the concentration of the nucleic acid from the unknown sample is estimated without mixing a labeling substance thereto. The principle is explained using FIG. 21 and FIG. 22. In all the following Examples, the labeling substance is not mixed with the nucleic acid from the unknown sample (105).

The target nucleic acid A of FIG. 21 is the same as the nucleic acid from the unknown sample (105 in FIG. 10) and the labeled probe-complementary nucleic acid is mixed in Example 1, the labeled molecule is intentionally not mixed in this example. This target nucleic acid A is a nucleic acid which is expected to hybridize with a probe, and is a single-stranded nucleic acid which will compete with the labeled probe-complementary nucleic acid. Examples of these types of nucleic acid include RNA, single-stranded cDNA synthesized from RNA, DNA synthesized by asymmetrical PCR, etc.

In FIG. 21, the black arrows written in the nucleic acid represent the 5′to 3′ direction of the nucleic acid, and two white arrows show competition in hybridization reaction.

On the other hand, in FIG. 22, the unknown nucleic acid B is a nucleic acid which is expected to hybridize with the labeled probe-complementary nucleic acid, and two white arrows show competition that will occur in hybridization reaction.

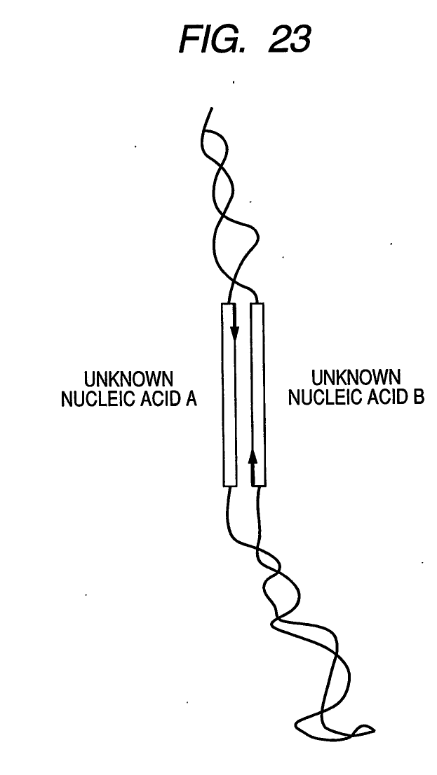

The relation between the target nucleic acid A of FIG. 21 and the nucleic acid B of FIG. 22 is shown in FIG. 23. When usual PCR or the like is performed for an sample, the nucleic acid pair as shown in FIG. 23 is the main component of nucleic acid from unknown sample 105 of FIG. 10. In this case, competitive reactions of FIG. 21 and FIG. 22 will occur simultaneously.

As explained above, the competitive reaction occurs in the hybridization step (106) of FIG. 10, and, as a result, the hybridized complementary nucleic acid (101) and probe DNA molecule as shown in FIG. 11 are separated. Consequently, a certain change arises in the fluorescence intensity on the microarray. By measuring the difference in the fluorescence intensity before and after the reaction with unknown nucleic acid, the separated labeled complementary nucleic acid is calculated for quantification of the nucleic acid (105) derived from the unknown sample. On this occasion, as shown in

FIG. 22, the separated labeled nucleic acid hybridizes to the nucleic acid B (the complementary nucleic acid of the nucleic acid A that hybridized to the probe).

This prevents annealing of the nucleic acids A and B, and is expected to enhance the hybridization on the microarray.

Actually, the labeled complementary nucleic acids (101) bound to probe DNA will peal off a little in the hybridization step 106 even under non-competitive conditions. Thus, a probe nucleic acid having a sequence which is supposed hardly contained in the nucleic acid from the unknown sample may be spotted on the DNA microarray as a positive control, to which a labeled complementary nucleic acid that hybridizes to the control probe specifically is applied in advance to estimate the pealing off amount of labeled strands under non-competitive conditions. Based on that, the amount of the nucleic acid in an unknown sample can be estimated by measuring the amount of the released separated labeling substance under competitive conditions.

### Example 4

In this Example, a series of consecutive dilutions is used for quantification of the nucleic acid (105) from the unknown sample in FIG. 10.

FIG. 5 schematically represents the results of quantification of the probe-bound labeled stand after the hybridization step (106) with various concentrations of the sample nucleic acid (105) in FIG. 10. FIG. 5 shows that in the range of 1,000-fold to 100-fold dilution of the sample nucleic acid (105), the labeled complementary nucleic acid (101) is hardly released, and the concentration of the detected (remained) labeled strand is high. That is, the result is the same when the hybridization solution contains no nucleic acid that hybridizes to the probe is applied to the DNA microarray in a condition shown in FIG. 11. Examples of the measured intensity are shown below the drawing.

On the other hand, when the hybridization step 106 is performed using the sample nucleic acid (105) of FIG. 10 without dilution, the competitive hybridization reaction described in Example 2 occurs, and labeled complementary nucleic acid (101) is released. Consequently, the intensity of the detected (remained) label becomes very low. As a result, one can assume that the concentration of the sample nucleic acid is 1/100 to 1 times of the concentration of the labeled complementary nucleic acid (101) of FIG. 10. By preparing and using a finer dilution series such as 80-fold, 40-fold, and 20-fold dilutions based on the above result, one can estimate the concentration of the unknown nucleic acid more precisely.

### Example 5

As described in Example 1, it is possible to prepare the labeled complementary nucleic acid (101) of FIG. 10 of extremely high purity. On the other hand, usually the nucleic acid from the unknown sample (105) is isolated and then synthesized through biochemical reaction of several steps. Thus there is a possibility of contamination of impurities in the synthetic process, and there may be nucleic acid of different lengths and types.

For this reason, the purity of the labeled complementary nucleic acid (101) is higher than that of the sample nucleic acid, so that apparently the intensity due to hybridization is affected more strongly by the labeled complementary nucleic acid. Therefore, in the embodiments other than Example 1 where the quantity of the sample nucleic acid is estimated on the basis of reduction of the bound amount of the label, it may be necessary to make the quantity of the sample nucleic acid sufficiently large. In order to solve this problem, this Example illustrates how to detect sample nucleic acid of a rather small amount.

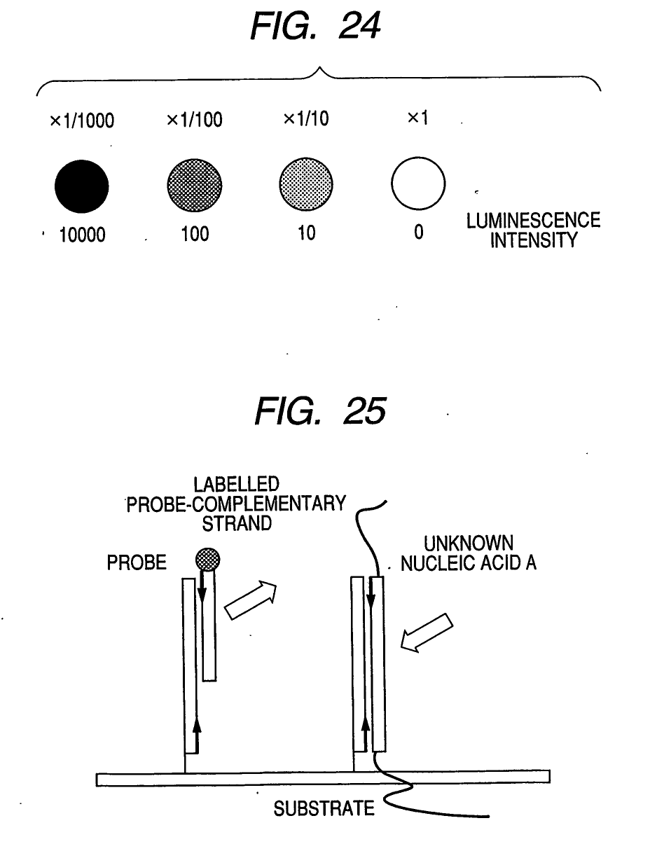

As shown in FIG. 25, the length of the labeled probe-complementary nucleic acid (101) in FIG. 10 is made shorter than the probe, for example, the probe nucleic acid is 20-mer, and the labeled probe-complementary nucleic acid is 10-mer. As a result, the hybridization reaction between the probe and the complementary nucleic acid becomes weak compared with the case in Examples 1 to 3 where the entire complementary nucleic acid is used. On the other hand, the target nucleic acid A in FIG. 3 is usually much longer than 20-mer, and contains a portion entirely complementary to the probe nucleic acid. Therefore, in FIG. 25, the hybridization indicated by the right white arrow between the probe and the target nucleic acid A is stable compared with the hybridization indicated by the left white arrow between the probe and the labeled probe-complementary nucleic acid. As a result, one can observe decrease in the detected (or remained) amount of the label as illustrated in FIG. 24 even when the concentration of the target nucleic acid A is rather low.

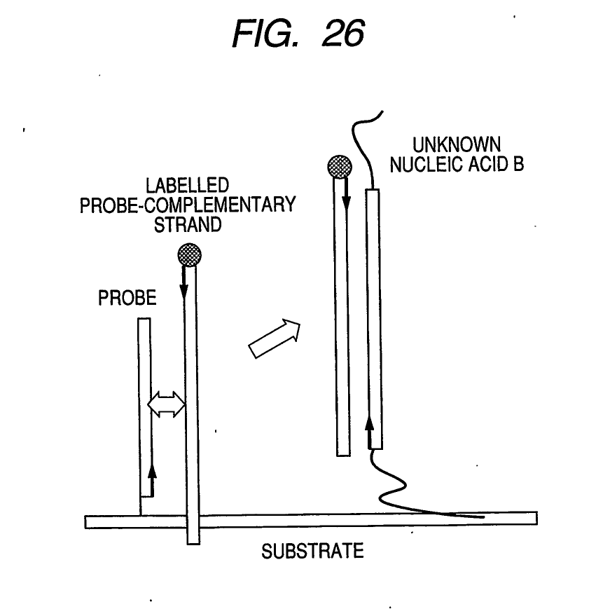

FIG. 26 shows a case where the labeled probe-complementary nucleic acid (101) is made longer than the probe. For example, the length of the probe is set to 20-mer and the labeled probe-complementary nucleic acid is set to 30-mer.

In this case, if the sequence of the extended portion is one expected to hybridize with the sample nucleic acid B, hybridization reaction between the labeled probe-complementary nucleic acid and the sample nucleic acid B becomes stronger. On the other hand, the length of the hybridizable portions of the probe and the labeled probe-complementary nucleic acid is still 20-mer.

Therefore, as shown in FIG. 26, the hybridization reaction indicated by the right white arrow between the labeled probe-complementary nucleic acid and the sample nucleic acid B becomes stronger than the hybridization reaction between the probe and the labeled probe-complementary nucleic acid indicated by the left white arrow, and the equilibrium shifts toward the release of the labeled probe-complementary nucleic acid increasing the chance in which the target nucleic acid A binds to the microarray. As a result, one can observe decrease in the detected (or remained) amount of the label as illustrated in FIG. 24 even when the concentration of the target nucleic acid A is rather low.

According to this embodiment, there is provided an advantage that the DNA microarray of high quality is guaranteed at a low cost. Conventionally, even when the probe spots contain DNA molecules in the same amount, to which the same number of the sample nucleic acid has bound by hybridization reaction, the measured values of the bound labeling molecules may differ. This has made it still more difficult to perform the quantification of the sample nucleic acid by using the DNA microarray. According to the process of this embodiment, the quantification of the probe DNA on the DNA microarray is made in advance of the hybridization experiment, a more precise experiment can be performed. Moreover, there are advantages that treatment of the analytes is much simplified and the quantitativity of the experiment system using the DNA microarray is further improved.

The present invention is not limited to the above embodiments and various changes and modifications can be made within the spirit and scope of the present invention. Therefore to apprise the public of the scope of the present invention, the following claims are made.

1. A process for treating a nucleic acid probe immobilized at a predetermined position on a substrate for nucleic acid detection, the process comprising the steps of:
preparing a labeled nucleic acid of a known sequence which contains a sequence complementary to the probe nucleic acid and can specifically bind to the probe, the labeled nucleic acid being not a target nucleic acid to be detected;
treating the immobilized probe on the substrate with the labeled nucleic acid to make the labeled nucleic acid bind to the probe.

2. The treating process according to claim 1 wherein the labeled nucleic acid is an artificial nucleic acid.

3. A process for detecting a nucleic acid using an nucleic acid probe being a specified nucleic acid immobilized at a known position on a substrate, the process comprising the steps of:
preparing a labeled nucleic acid of a known sequence which contains a sequence complementary to the probe nucleic acid and can specifically bind to the probe, the labeled nucleic acid being not a target nucleic acid to be detected;
treating the immobilized probe on the substrate with the labeled nucleic acid to make the labeled nucleic acid bind to the probe
reacting a target nucleic acid with the probe on the substrate; and
detecting an amount of the label nucleic acid attached to the immobilized probe.

4. A process for detecting a target nucleic acid comprising the steps of:
providing a nucleic acid probe being a predetermined nucleic acid immobilized at a predetermined position on a substrate;
providing a solution containing a sample nucleic acid and a labeled nucleic acid of a known sequence which contains a sequence complementary to the probe nucleic acid and can specifically bind to the probe, the labeled nucleic acid being not a target nucleic acid to be detected;
contacting the solution to the probe to allow binding of the nucleic acids in the solution to the immobilized probe; and
detecting an amount of the labeled nucleic acid attached to the immobilized probe nucleic acid,
wherein the target nucleic acid bound to the probe is detected based on the detected amount of the labeled nucleic acid.

5. A process for analyzing a nucleic acid concentration using an immobilized probe nucleic acid being a specific nucleic acid immobilized at a predetermined position on a substrate, the process comprising the steps of
(1) preparing a solution containing a labeled nucleic acid of a known sequence which contains a sequence complementary to the probe nucleic acid and can specifically bind to the probe in a predetermined concentration and also a nucleic acid derived from a sample;
(2) contacting the solution with the immobilized probe for hybridization; and
(3) detecting an amount of the labeled nucleic acid attached to the probe, wherein a concentration of the sample-derived nucleic acid in the solution is estimated based on a decrease in the amount of the labeled nucleic acid attached to the probe obtained in the step (3) compared to an amount of the labeled nucleic acid attached to the probe when hybridization is carried out with a solution containing the labeled nucleic acid at a predetermined concentration but not containing the nucleic acid derived from the sample.

6. A kit for detecting a target nucleic acid in a sample comprising:
an immobilized nucleic acid probe being a specific nucleic acid immobilized at a predetermined position on a substrate; and
a solution containing a labeled nucleic acid of a known sequence which contains a sequence complementary to the probe nucleic acid and can specifically bind to the probe,
wherein the solution is used as a mixture with a solution containing the sample.

7. A process for analyzing a nucleic acid concentration using an immobilized probe nucleic acid being a specific nucleic acid immobilized at a predetermined position on a substrate, wherein the process comprises steps of:
introducing a first solution containing a target nucleic acid to be determined derived from a sample into a chamber in which the immobilized probe nucleic acid has been arranged;
introducing a second solution which contains a labeled nucleic acid of a known sequence which contains a sequence complementary to the probe nucleic acid and can specifically bind to the probe so that the both solution may be mixed with each other; and
detecting the amount of the label of the labeled nucleic acid bound to the immobilized probe nucleic acid, wherein
the concentration of the target nucleic acid in the mixed solution is estimated based on the correlation between the amount of the second solution introduced into the chamber and a change in the amount of the label attached to the immobilized probe after the introduction of the second solution.

8. The process according to claim 7 wherein the detection of the amount of the label is carried out with a confocal microscope.

9. An immobilized probe nucleic acid for nucleic acid detection wherein a probe nucleic acid is immobilized at a predetermined position on a substrate and a labeled nucleic acid, to which a complementary nucleic acid specifically bindable to the probe nucleic acid has been bound by hybridization.

10. The immobilized probe nucleic acid according to claim 9, wherein at least 60% of the probe nucleic acid immobilized on the substrate is bound to the labeled nucleic acid.

11. A substrate containing an immobilized probe nucleic acid according to claim 8 immobilized thereon.

12. A process for detecting a nucleic acid comprising the steps of:
providing an immobilized probe nucleic acid being a specific nucleic acid immobilized at a predetermined position on a substrate, to which a labeled nucleic acid of a known sequence which contains a sequence complementary to the probe nucleic acid and can specifically bind to the probe is binding by hybridization at a predetermined amount;
contacting a solution containing a target nucleic acid with the immobilized probe nucleic acid for hybridization; and
determining an amount of the labeled nucleic acid attached to the immobilized probe nucleic acid, wherein
the target nucleic acid bound to the immobilized probe nucleic acid is determined by comparison of the predetermined amount of the labeled nucleic acid attached to the immobilized probe nucleic acid and the amount of the labeled nucleic acid determined in the determination step.

13. A process for analyzing a nucleic acid concentration, wherein the process comprises steps of:
(1) providing an immobilized nucleic acid probe by immobilizing a specific nucleic acid at a predetermined position on a substrate;
(2) binding a labeled nucleic acid, which has a complementary nucleic acid specifically bindable to the immobilized probe nucleic acid, to the immobilized probe nucleic acid by hybridization;
(3) contacting a solution containing a target nucleic acid with the probe for hybridization; and
(4) detecting an amount of the labeled nucleic acid bound to the probe,
wherein the concentration of the nucleic acid derived from the sample in the solution is estimated by comparison of an amount of the labeled nucleic acid bound to the probe in the preliminary hybridization step (2) and the amount of the labeled nucleic acid detected in the detection step (3).

14. A process for analyzing a nucleic acid to determine gene polymorphism comprising steps of:
(1) providing nucleic acid probes by immobilizing nucleic acids corresponding to all alleles of a target gene polymorphism at predetermined positions on a substrate;
(2) contacting a plurality of labeled nucleic acids having complementary sequence to the probes respectively and able to bind thereto specifically, and an unlabeled unknown nucleic acid derived from a sample to the probes for hybridization; and
(3) detecting an amount of the labeled nucleic acid bound to the probe nucleic acids, wherein gene polymorphism of the nucleic acid derived from the sample is determined based on the balance of the amounts of the labeled nucleic acids bound to the probes.

15. The process according to claim 14, wherein the gene polymorphism is SNP (Single Nucleotide Polymorphism).

16. The process according to claim 14, wherein the labeled complementary nucleic acids each has a predetermined number of labels.

17. The process according to claim 14, wherein the labeled complementary nucleic acids each has one label.

18. The process according to claim 14, wherein the labels are different according to the alleles of gene polymorphism.

19. The process according to claim 14, wherein the labels are fluorescent labels having different emission wavelengths according to the alleles of gene polymorphism.

20. The process according to claim 14, wherein the step (2) further comprises in sequence the steps of:
(i) providing a solution containing a labeled nucleic acid of a known sequence which contains a sequence complementary to the probe nucleic acid and can specifically bind to the probe and an unlabeled unknown nucleic acid derived from a sample; and
(ii) contacting the solution to the probe for hybridization.

21. The process according to claim 14, wherein the step (2) comprises in sequence the steps of:
(i) contacting a solution containing labeled nucleic acids of known sequences which contain sequences complementary to the probe nucleic acids and can specifically bind to the probe nucleic acids for hybridization;
(ii) detecting the amount of the label of the labeled nucleic acid bound to the each probe;
(iii) contacting the unlabeled unknown nucleic acid with the probe nucleic acids for hybridization; and
(iv) detecting the amount of the labeled nucleic acid bound to the each probe nucleic acid.

22. The process according to claim 14, wherein probes corresponding to all alleles of the gene polymorphism are immobilized together at a single location by a single spotting.

23. The process according to claim 14, wherein probes for different alleles of the gene polymorphism are immobilized at different locations on the substrate.

24. A kit for identifying gene polymorphism comprising:
a substrate with immobilized nucleic acids on which all the allelic nucleic acids to be examined are immobilized at known locations, respectively, on the substrate to specify gene polymorphism at the respective locations; and
labeled nucleic acids of known sequences each containing a sequence complementary to one of the allelic nucleic acids and can specifically bind thereto.

25. A process for identifying a gene polymorph comprising the steps of:
providing at least two nucleic acids of different polymorphs of a gene or complementary nucleic acids thereof immobilized at predetermined positions on the substrate;
binding at least two labeled nucleic acids known to specifically bind to the immobilized nucleic acids to the immobilized nucleic acids by hybridization;
binding an unknown and unlabeled nucleic acid to the immobilized nucleic acid by hybridization; and
identifying the polymorphism of the unknown nucleic acid based on a reduction of an amount of the labeled nucleic acid bound to the immobilized nucleic acids caused by binding of the unknown nucleic acid to the immobilized nucleic acid.
